SEQUENCE LISTING (5 sequences)

SEQ ID NO: 1 - DNA, 203 bp, homo sapien
  misc_feature (1)..(203): Exosome (hsa_circ_0004771)
gaagtgtttggattgtgagctatttcagaactgttctcaggactcattattttaacatttgggagaaacacagccagaagatgcacacttgactgaaggaggacagggaatctgaagactccggatgacatcagagctacttttcaacagccttctcaattttctttctcagaaagcagaggctcagagcttggagacagacg

SEQ ID NO: 2 - DNA, 20 bp, Artificial Sequence
  primer hsa_circ_0004771-F
ctccggatgacatcagagct

SEQ ID NO: 3 - DNA, 20 bp, Artificial Sequence
  primer hsa_circ_0004771-R
tctggctgtgtttctcccaa

SEQ ID NO: 4 - DNA, 20 bp, Artificial Sequence
  forward primer for GAPDH
gcaccgtcaaggctgagaac

SEQ ID NO: 5 - DNA, 21 bp, Artificial Sequence
  reverse primer for GAPDH
ggatctcgctcctggaagatg

(19) United States
(12) Patent Application Publication
(10) Pub. No.: US 2022/0154279 A1
(43) Pub. Date: May 19, 2022

(54) USE OF SERUM EXOSOMAL HSA_CIRC_0004771 IN PREPARING REAGENTS FOR DIAGNOSIS OF ALCOHOL DEPENDENCE

(71) Applicants: SUN YAT-SEN UNIVERSITY, Guangzhou (CN); SUN YAT-SEN MEMORIAL HOSPITAL, SUN YAT-SEN UNIVERSITY, Guangzhou (CN)

(72) Inventors: Ying PENG, Guangzhou (CN); Yunyun LIU, Guangzhou (CN)

(73) Assignees: SUN YAT-SEN UNIVERSITY, Guangzhou (CN); SUN YAT-SEN MEMORIAL HOSPITAL, SUN YAT-SEN UNIVERSITY, Guangzhou (CN)

(21) Appl. No.: 17/600,110

(22) PCT Filed: Jun. 19, 2020
(86) PCT No.: PCT/CN2020/097041

§ 371 Date: Sep. 30, 2021

(30) Foreign Application Priority Data
Apr. 22, 2019 (CN) 201910323834.9

(51) Int. Cl. C12Q 1/6883; C12Q 1/6851; C12Q 1/6806

(52) U.S. Cl. CPC C12Q 1/6883; C12Q 2600/112; C12Q 1/6806; C12Q 1/6851

(57) ABSTRACT

A method of using a serum exosomal hsa_circ_0004771 in preparing a reagent for the diagnosis of alcohol dependence is provided. Through study on the changes in expression levels of serum exosomal circRNA in patients with alcohol dependence (AD), a useful circRNA has been discovered and its effectiveness on clinical detection of AD has been validated. The exosomal hsa_circ_0004771 is useful for the diagnosis of AD as its expression level is related to the severity of AD, and therefore can be used as a biomarker in the diagnosis and severity assessment of AD. Development and application of the serum-derived circRNA biomarker and kit makes AD diagnosis much more convenient and simply, allowing clinicians to rapidly and accurately identify patients' conditions and providing improved clinical therapeutic effects; it also contributes to the discovery of novel potential drugs.

### CROSS REFERENCE TO THE RELATED APPLICATIONS

This application is the national phase entry of International Application No. PCT/CN2020/097041, filed on Jun. 19, 2020, which is based upon and claims priority to Chinese Patent Application No. 201910323834.9, filed on Apr. 22, 2019, the entire contents of which are incorporated herein by reference.

### SEQUENCE LISTING

The instant application contains a Sequence Listing which has been submitted in ASCII format via EFS-Web and is hereby incorporated by reference in its entirety. Said ASCII copy is named GBKY069_Sequence Listing.txt, created on 09/10/2021 and is 1,665 bytes in size.

### TECHNICAL FIELD

The present invention relates to the technical field of biology, and particularly relates to a serum exosomal, circular biomarker hsa_circ_0004771, primers and kits for detecting the biomarker, and use of the biomarker, primers and kits in the diagnosis and severity assessment of alcohol dependence.

### BACKGROUND

Alcohol dependence (AD) is defined as patients' inability to control alcohol consumption, which has a negative impact on physical and mental health and interpersonal relationships. AD is very common in developed and developing countries, with a prevalence of about 10%, of the global population. Although efforts have been made to develop methods for the diagnosis of AD based on DSM-IV/V criteria, it is reported that less than 15% of patients receive diagnosis and treatment. Therefore, development of new biomarkers for the detection of AD has aroused great interest of neuroscientists.

Exosomes are spherical vesicles with a diameter of 30-100 nm, and have largely been recognized for their role in intercellular communication by transporting functional proteins and nucleic acids to recipient cells. Recent studies show that exosomes may be produced by a wide range of cell types in the central nervous system, including oligodendrocytes, Schwann cells, microglia and astrocytes. Exosomes actively participate in synaptic plasticity, signal transduction, neuroinflammation and degeneration, involving a wide range of normal and pathological processes, which is consistent with their role in the occurrence and development of many central nervous system diseases such as infections, Parkinson's disease, Alzheimer's disease, amyotrophic lateral sclerosis, and stroke. However, there are little studies on the use of exosomes as biomarkers for the detection of alcohol dependence.

Circular RNAs (circRNAs) are a new type of RNA widely found in eukaryotes from Caenorhabditis elegans to humans, characterized by their evolutionary conservation; their 3′ and 5′ ends are not free but covalently joined to give a closed ring. Formed by a specific pattern called “backsplicing”, circRNAs may be derived from coding and noncoding exons (ecircRNAs), introns (ciRNAs), exons-introns (EIciRNAs), or 5′ and 3′ untranslated regions (UTRs). Circular RNAs are abundantly found in organisms and are expressed in cell type, tissue type and stage-specific patterns. Although circRNAs have been detected in various tissues, they have been found to be more abundant in the brain. They also play a key role in the proliferation and differentiation of neurons.

In view of the above, dysregulation of circRNAs in the central nervous system may eventually lead to various diseases. In addition, circRNAs have also been reported to be related to other neuropsychological diseases, such as major depression, schizophrenia, Duchenne muscular dystrophy, and glioma. We presumed that circRNAs may serve as biomarkers and therapeutic targets for AD, but further studies are required. One object of the present disclosure is to find a novel circular biomarker for AD diagnosis from serum exosomes through evaluating change of circRNAs expression.

### SUMMARY

A first object of the present invention is to provide primers for specific detection of a serum exosomal biomarker hsa_circ_0004771, wherein the primers comprise:

hsa_circ_0004771-F:
(SEQ ID NO: 2)
5′-CTCCGGATGACATCAGAGCT-3′,
and
hsa_circ_0004771-R:
(SEQ ID NO: 3)
5′-TCTGGCTGTGTTTCTCCCAA-3′.

A second object of the present invention is to provide the serum exosomal biomarker hsa_circ_0004771, wherein the biomarker hsa_circ_0004771 has a nucleic acid sequence of SEQ ID NO.1.

A third object of the present invention is to provide use of the above primers in preparing a reagent for the diagnosis and severity assessment of alcohol dependence.

A fourth object of the present invention is to provide use of the serum exosomal biomarker hsa_circ_0004771 as a biomarker in preparing a reagent for the diagnosis and severity assessment of alcohol dependence.

A fifth object of the present invention is to provide use of a reagent for determining a concentration of the biomarker hsa_circ_0004771 in serum exosomes in preparing a kit for the diagnosis and severity assessment of alcohol dependence.

A sixth object of the present invention is to provide a kit for the diagnosis and severity assessment of alcohol dependence, wherein the kit comprises the above primers for specific detection of the serum exosomal biomarker hsa_circ_0004771.

Furthermore, the kit further comprises all reagents necessary for isolating exosomes from serum, isolating RNA from the exosomes, reverse transcription, and quantitative fluorescence PCR (polymerase chain reaction).

The present invention has the following beneficial effects: The circular RNA hsa_circ_0004771 has been discovered for the first time in serum exosomes. Expression levels of the serum exosomal hsa_circ_0004771 in healthy volunteers and patients with alcohol dependence (AD) determined by QRT-PCR showed that, the expression level in AD patients were significantly higher than that in the healthy volunteers (p<0.001), suggesting that the exosomal hsa_circ_0004771 is useful for the diagnosis of alcohol dependence. In addition, analysis showed that the hsa_circ_0004771 level was positively correlated with both SADQ and ADS scores (r=0.8484 and 0.8616), suggesting that the biomarker hsa_circ_0004771 relates to AD severity, and thereby it is possible that the hsa_circ_0004771 is a sensitive biomarker for distinguish AD patients from non-patients. Furthermore, development and application of the serum-derived circRNA biomarker and kit makes AD diagnosis much more convenient and simply, allowing clinicians to rapidly and accurately identify patients' conditions and providing improved clinical therapeutic effects. It also contributes to the discovery of novel potential drugs.

### BRIEF DESCRIPTION OF THE DRAWINGS

FIGS. 1A-IC show the results of identification of serum exosomes. FIG. 1A includes representative TEM images of the exosomes (bar=100 nm). FIG. 1B shows the expression levels of CD63, TSG101, and HSP90B1 in serum exosomes determined by Western blot. FIG. 1C shows the concentration and size distribution measured by NTA.

FIG. 2 shows the expression levels of the serum exosomal hsa_circ_0004771 in healthy controls and AD patients.

FIGS. 3A-3C show the correlations between the expression level of hsa_circ_0004771 and AD severity. FIG. 3A shows the correlation between expression level of hsa_circ_0004771 and SADQ score. FIG. 3B shows the correlation between expression level of hsa_circ_0004771 and ADS score. FIG. 3C shows the area under curve (AUC) in the ROC analysis of the serum exosomal hsa_circ_0004771.

### DETAILED DESCRIPTION OF THE EMBODIMENTS

The following examples are to further illustrate the present invention, but not to limit the present invention.

### Example 1

1. Participant Criteria

The study included 37 age-matched healthy volunteers (non-AD control) and 60 AD patients. All patients were admitted to the Department of Neurology of the Sun Yat-sen Memorial Hospital from December 2016 to December 2018. Inclusion criteria for AD patients: (1) aged 18-80 years; (2) meeting the criteria of probable AD defined by the DSM-IV (Diagnostic and Statistical Manual of Mental Disorders); (3) still drinking alcohol within a week before admission; (4) being able to complete basic interviews (MMSE score >10); (5) willing to provide blood samples for exosomes and circular RNA analysis.

The age-match non-AD control group consisted of healthy volunteers recruited from communities near Sun Yat-sen Memorial Hospital through advertising. In order to meet the criteria as a control, the volunteers were either abstainers or social drinkers with daily alcohol consumption no more than 25 g recommended by the Chinese Dietary Guidelines (2016). Most importantly, the volunteers must be non-alcohol dependent with their AUDIT-C score less than 5 (33).

In order to minimize the impact of unknown contaminants on our results, AUD subjects and volunteers would be excluded from the study if they met one or more of the following criteria: (1) having a diagnosis of drug dependence other than nicotine dependence; (2) having a current or past diagnosis of psychiatric comorbidity (except mild anxiety or depression) such as schizophrenia or bipolar disorder; (3) having a neurodegenerative comorbidity such as Parkinson's disease or Alzheimer's disease; (4) having a significant physical disease such as diabetes, renal insufficiency, infarction, liver cirrhosis, severe infectious disease, cancer; (5) HIV infected; (6) pregnant; (7) refusing to provide blood samples and/or written informed consent.

The study was given ethics committee approval by Sun Yat-sen Memorial Hospital under number SYSEC-KY-KS-2019007, and written informed consent was obtained from all participants.

All continuous data are expressed as median (range). Categorical variables are expressed as values (percentages).

2. Collection of Clinical Specimens

10 mL blood samples were collected from the healthy volunteers and AD patients for the isolation of exosomes. In order to isolate the exosomes, serum samples were centrifuged at 3000 g for 15 minutes at 4° C. to remove apoptotic bodies and thereby supernatants were collected.

3. Isolation of Serum Exosomes

The supernatants were delivered to new tubes where exosomes were isolated using the ExoQuick reagent (01.SBI.EXOQ5A-1, Sigma, USA) by the following steps:

(1) 500 μL of supernatant was pipetted to a new EP tube (Eppendorf tube) and centrifuged at 3000 rcf for 15 minutes to remove cells or cell debris, and the resultant supernatant was delivered to another autoclaved EP tube.

(2) The supernatant was then mixed with 250 μL of thrombin. The mixture was incubated at 37° C. for 15 minutes and then centrifuged at 10000 rpm at room temperature for 15 minutes to give completely separated supernatant and precipitate.

(3) The precipitate was removed and the supernatant was collected to give a “serum-like” liquid. 600 μL of the “serum-like” liquid was delivered to another centrifuge tube followed by the addition of 150 μL of ExoQuick reagent. The mixture was incubated at 4° C. for 30 minutes and then centrifuged at 1500 rcf at room temperature for 30 minutes. A light yellow or white precipitate was observed at the bottom of the tube.

(4) After removal of the supernatant, the precipitate was centrifuged at 1500 rcf for another 5 minutes, and then the supernatant liquid was removed as much as possible. A proper amount of PBS was then added to resuspend the precipitate until it is completely dissolved. The resultant exosome solution was stored at 80° C. for later use.

4. Identification of Exosomes

(1) Transmission Electron Microscope (TEM)

Morphology of exosomes was inspected using a Hitachi HT7700 transmission electron microscope; 20-40 μL of solutions were placed onto carbon-coated Formvar grids and allowed to settle for 10 minutes, and then stained by phosphotungstate (pH 6.8) for 5 minutes. TEM images of the samples were captured.

(2) Nanoparticle Tracking Analysis (NTA)

Size distribution and concentration of the exosomes were analyzed by a NanoSight NS300 (NanoSight Ltd., Amesbury, UK). The exosomes were diluted with particle-free PBS before placed into the sample chamber. Analysis was carried out with an NTA software (version 2.3, NanoSight Ltd).

(3) Immunoblotting Analysis

Exosomal markers were examined by Western blot. After lysed by RIPA buffer (Pierce, Rockford, Ill., USA), the samples were subjected to separation by gel electrophoresis (12% sodium dodecyl sulphate-polyacrylamide). The proteins were then transferred to PVDF membranes, which were then blocked by 5% bovine serum albumin. Membranes were separately incubated with mouse anti-CD63 antibody (Abcam, 1:400), anti-TSG101 antibody (Abcam, 1:1000), rabbit polyclonal anti-HSP90B1 antibody (Cell Signaling Technology, 5:10000), or mouse monoclonal anti-β-actin antibody (Abcam, 2.5:10000). Protein analysis was carried out using enhanced chemiluminescence (Pierce, Rockford, Ill., USA).

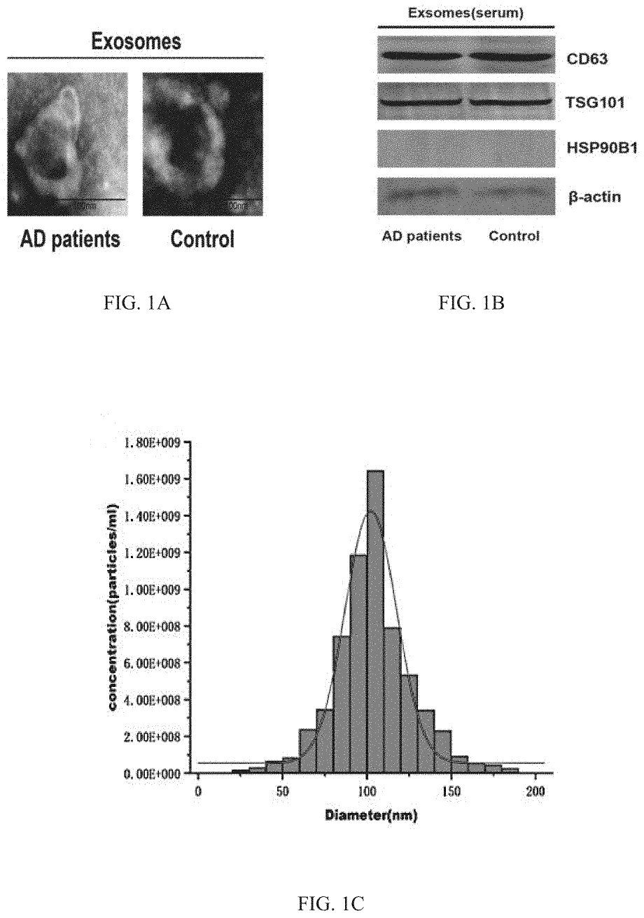

FIGS. 1A-IC show the results of identification of serum circulating exosomes by TEM, NTA, and Western blot. TEM inspection (FIG. 1A) showed that the exosomes were irregular spheres with a double-layer membrane structure and a size range of 50-100 nm. The NTA analysis (FIG. 1C) showed that the exosomes exhibited a particle size peak at 105 nm with a relatively narrow distribution. Western blot analysis validated the presence of CD63 and TSG101 and the absence of HSP90B1 in the exosomes, suggesting that the exosomes were purified (FIG. 1B). The above results indicate that the exosomes isolated from serum exhibited exosomal characteristics.

5. Isolation of Exosomal RNA

(1) 200 μL of serum exosomes collected from the AD patients (or healthy volunteers) in the above steps were mixed with 1 mL of TRIZOL reagent and repeatedly pipetted to lyse the exosomes. After homogenization, the samples were incubated at 15-30° C. for 5 minutes to allow complete dissociation of nucleic acid-protein complex.

(2) To each sample (which was homogenized by 1 mL of TRIZOL reagent) was added 0.2 mL of chloroform, then the tubes were shaken vigorously for 15 seconds. The samples were incubated at 15-30° C. for 2-3 minutes, and centrifuged at 4° C. at 12000 rcf for 15 minutes. After the centrifugation, the mixtures were separated into a lower red phenol-chloroform phase, and middle and upper colorless water phases.

(3) The water phases were transferred to new centrifuge tubes and added with 0.5 mL of isopropanol. The mixtures were then incubated at 15-30° C. for 10 minutes, and centrifuged at 4° C. at 12000 rcf for 10 minutes. After removal of supernatants, the precipitated RNA was washed by shaken in at least 1 mL of 75% ethanol aqueous solution. The mixtures were then centrifuged at 4° C. at 7500 rcf for 5 minutes. After removal of the ethanol aqueous solution, the precipitated RNA was air-dried for 5-10 minutes and then dissolved in 50 μL of DEPC-treated water, stored for later use.

(4) RNA concentration and purity were assessed using Nano Drop® ND-2000.

6. Synthesis of Exosomal cDNA

(1) Reagents necessary for reverse transcription of circRNA were thawed, inverted slightly to allow for even mixing, centrifuged briefly, and placed on ice for later use.

(2) Preparation of circRNA reverse transcription system: The reagents were added into RNase-free tubes (pre-cooled on ice) to a total volume of 20 μL (see Table 1 for the reaction system).

TABLE 1
RT-PCR System

| Component | Volume | Final Concentration |
|---|---|---|
| 5 × Prime Script Buffer | 4 μL | 1× |
| Random 6 mers (100 μM) | 4 μL | |
| RNA | 10 μL | 800 ng |
| Prime Script RT Enzyme Mix I | 1 μL | |
| dd H2O (RNase/DNase free) | 1 μL | |

(3) Reverse transcription: Conditions for reverse transcription: 37° C. (15 min)→85° C. (5 s). Once the reaction was complete, the resultant cDNA was stored at −80° C. or immediately subjected to quantitative PCR

7. Real-Time Quantitative Fluorescence PCR

(1) Primers

Sequences of primers for hsa_circ_0004771 and internal reference GAPDH are as shown in Table 2.

TABLE 2
Primer Sequences

| | | |
|---|---|---|
| hsa_circ_0004771 | Forward | 5′-CTCCGGATGACATCAGAGCT-3′ |
| | | (SEQ ID NO: 2) |
| | Reverse | 5′-TCTGGCTGTGTTTCTCCCAA-3′ |
| | | (SEQ ID NO: 3) |
| GAPDH | Forward | 5′-GCACCGTCAAGGCTGAGAAC-3′ |
| | | (SEQ ID NO: 4) |
| | Reverse | 5′- |
| | | GGATCTCGCTCCTGGAAGATG-3′ |
| | | (SEQ ID NO: 5) |

(2) The SYBR Premix Ex TaqTMII Tli RNaseH plus (RR820A) kit was used in this protocol. The reagents were thawed and mixed on ice according to Table 3 to give the reaction solutions.

TABLE 3
PCR System

| Component | Volume | |
|---|---|---|
| SYBR Premix Ex Taq II (Tli RNaseH Plus) | 10 | μL |
| PCR Forward Primer | 1 | μL |
| PCR Reverse Primer | 1 | μL |
| c DNA | 2 | μL |
| Rox II | 0.4 | μL |
| d H2O | 5.6 | μL |

(3) After the reagents were evenly mixed, the reaction solutions were added to 96-well PCR plates which were then sealed with films and centrifuged at 2000 rcf for 2 minutes.

(4) The qRT-PCR was then carried out according to the conditions listed in Table 4.

TABLE 4
PCR Cycle Conditions

| Number of Cycles | Step | Temperature | Time |
|---|---|---|---|
| 1 | Initial denaturation | 95° C. | 30 sec |
| 50 | Extension | 95° C. | 5 sec |
| | | 55° C. | 34 sec |

(5) Melt curve analysis was carried out according to the reagent requirement right after the PCR reaction was complete. Relative quantification (RQ) was performed with the obtained Ct value using the 2-ΔΔCt method, wherein, ΔCt=Ct (gene of interest)−Ct (reference gene), ΔΔCt=ΔCt (treated sample)−ΔCt (control sample), and RQ=2-ΔΔCt. The relative quantification refers to the change in gene expression of a given circRNA in a sample relative to another reference sample, wherein “RQ>1” indicates an increased expression of the circRNA of interest, while “RQ<1” indicates a decreased expression. It is generally considered that, when the 2-ΔΔCt method was used for relative quantification, an RQ value above 2 or below 0.5 indicates statistical significance between the data.

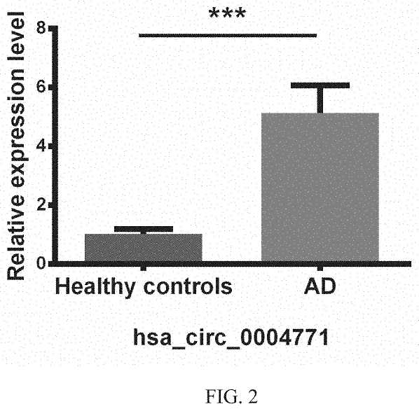

Expression levels of serum exosomal hsa_circ_0004771 in the healthy controls and the AD patients assessed by qRT-PCR were as shown in FIG. 2. It can be concluded from FIG. 2 that the hsa_circ_0004771 expression level in the AD patients was significantly higher than that in the healthy controls (p<0.001), suggesting that the exosomal hsa_circ_0004771 is useful for the diagnosis of alcohol dependence.

8. Statistics of Clinical Cases

(1) Analysis on Clinical Data of Patients with Alcohol Dependence

Clinical data analysis included case number, name, gender, age, years of drinking, alcohol consumption, SADQ score, and ADS score, as listed in Table 5.

TABLE 5
Clinical data of AD patients and healthy volunteers

| | | Healthy | | |
|---|---|---|---|---|
| | AD patients | volunteers | t | p |
| Average age | 45.50 ± 6.070 | 46.67 ± 6.927 | 0.1267 | 0.9017 |
| Male (cases) | 5 | 5 | N/A | N/A |
| Female (cases) | 1 | 1 | N/A | N/A |
| Years of drinking | 25.33 ± 4.224 | N/A | N/A | N/A |
| Daily consumption | 273.8 ± 33.19 | N/A | N/A | N/A |
| (mL) | | | | |
| SADQ | 34.83 ± 3.859 | N/A | N/A | N/A |
| ADS | 38.67 ± 2.155 | N/A | N/A | N/A |

(2) Analysis of the Correlation Between Clinical Data and Expression Levels of hsa_circ_0004771

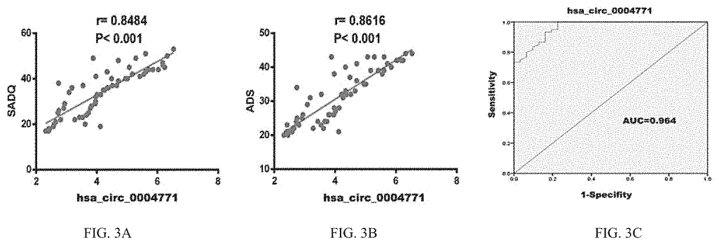

In order to assess the correlation between the serum exosomal hsa_circ_0004771 and AD severity, the correlation between the hsa_circ_0004771 level and SADQ and ADS scores was analyzed, wherein the hsa_circ_0004771 level in AD patients was determined by qRT-PCR, and AD severity was assessed by SADQ and ADS. We found that the level of exosomal hsa_circ_0004771 was positively correlated with the two scores (r=0.8484 and 0.8616; FIGS. 3A and B), suggesting that hsa_circ_0004771 level is related to the severity of AD. In order to further determine whether hsa_circ_0004771 is useful for the diagnosis of AD patients, ROC analysis was carried out on 60 AD cases and 37 healthy cases, wherein the area under curve (AUC) of the serum exosomal hsa_circ_0004771 was 0.964 (95% CI: 0.932-0.997, p<0.001; FIG. 3C), suggesting that the hsa_circ_0004771 may be used as a sensitive biomarker for distinguish AD patients from non-patients.

The above are only preferred embodiments of the present invention. It should be noted that the above preferred embodiments should not be regarded as limiting the present invention, and the scope of the present invention should be subject to the scope defined by the claims. For those of ordinary skill in the art, without departing from the spirit and scope of the present invention, several improvements and modifications can be made, and these improvements and modifications should also fall within the scope of the present invention.

We claim:

1. A pair of primers for specific detection of a serum exosomal biomarker having a nucleic acid sequence as set forth in SEO ID NO: 1, wherein the pair of primers comprise:
hsa_circ_0004771-F:
5′-CTCCGGATGACATCAGAGCT-3′, as set forth in SEQ ID
NO: 2,
and
hsa_circ_0004771-R:
5′-TCTGGCTGTGTTTCTCCCAA-3′, as set forth in SEQ ID
NO: 3.

2. (canceled)

3. (canceled)

4. A method for the diagnosis and severity assessment of alcohol dependence, comprising the step of using the pair of primers of claim 1 to perform the specific detection of the serum exosomal biomarker having the nucleic acid sequence as set forth in SEQ ID NO: 1.

5. The method according to claim 4, comprising the step of determining a concentration of the serum exosomal biomarker in serum exosomes.

6. A kit for the diagnosis and severity assessment of alcohol dependence, wherein the kit comprises the pair of primers of claim 1 for the specific detection of the serum exosomal biomarker having the nucleic acid sequence as set forth in SEQ ID NO: 1.

7. The kit according to claim 6, further comprising all reagents necessary for isolating exosomes from serum, isolating RNA from the exosomes, reverse transcription, and quantitative fluorescence PCR.
